(19) United States
(12) Patent Application Publication
(10) Pub. No.: US 2019/0323095 A1
(43) Pub. Date: Oct. 24, 2019

(54) COMPOSITIONS AND METHODS FOR ABSOLUTE QUANTIFICATION OF REVERSE TRANSCRIPTASE ENZYMES

(71) Applicant: University of Virginia Patent Foundation, Charlottesville, VA (US)

(72) Inventors: Thomas P. Loughran, JR., Charlottesville, VA (US); Thomas Lynn Olson, Palmyra, VA (US); David J. Feith, Palmyra, VA (US)

(21) Appl. No.: 16/461,981

(22) PCT Filed: Dec. 1, 2017
(86) PCT No.: PCT/US2017/064296

§ 371 Date: May 17, 2019

Related U.S. Application Data
(60) Provisional application No. 62/429,284, filed on Dec. 2, 2016.

(51) Int. Cl. C12Q 1/70; C12Q 1/6806

(52) U.S. Cl. CPC C12Q 1/702; C12Q 1/6806

(57) ABSTRACT

The present application discloses compositions and methods utilizing a combination of Pert and ddPCR technologies that yield, for example, the unexpected result of absolute quantification of the presence of RT enzymes. The novel technology disclosed herein is referred to as ddPERT. This is an advance over prior technology such as PERT.

### PRIORITY

This application claims the benefit of priority from U.S. Provisional Patent Application No. 62/429,284, filed on Dec. 2, 2016, which is herein incorporated in its entirety by reference.

### BACKGROUND OF THE INVENTION

The product-enhanced reverse transcriptase (PERT) assay has been used to detect reverse transcriptase (RT) activity associated with retroviruses. The product-enhanced reverse transcriptase assay has been in use for some time and is the subject of multiple patents. The basis of this assay is reverse transcriptase PCR (RT-PCR). In typical RT-PCR the goal is to measure RNA levels and RT is added in excess to make DNA complementary to mRNA (cDNA) which is then amplified in the presence of specific chemistries that allow this amplification to be measured and relative quantification of mRNA species to be made. Both dsDNA detection with Sybr dyes (SG-PERT), as well as hydrolysis probe chemistries (F-PERT) have been described and used. This is standard quantitative real time PCR. By contrast, PERT assays supply RNA templates and other components to be mixed with viral or recombinant derived RT with the express desire of determining how many functional RT molecules are present. Reverse transcriptases are contained within retroviruses, and not mammalian cells or other viruse types, where they serve their natural function of converting the viral RNA genome into DNA so that it may be later integrated into host DNA

### SUMMARY OF INVENTION

The present application discloses compositions and methods utilizing a combination of PERT and ddPCR technologies that yield, for example, the unexpected result of absolute quantification of the presence of RT enzymes. The novel technology disclosed herein is referred to as ddPERT. This is an advance over prior technology such as PERT.

One embodiment provides a method to determine if a sample comprises a reverse transcriptase comprising: a) mix a sample with template RNA, along with nucleotides (deoxy triphosphates), primer (at least one primer), and DNA polymerase to generate a mixture (differing divalent cations can be used here, as different RTs perform better with different divalent cations (cation with a valence of 2; such as Mg²⁺, Ba²⁺, Ca²⁺, Cr²⁺, Cu²⁺, Fe²⁺, Pb²⁺, Hg₂²⁺, Hg²⁺, Sr²⁺, Sn²⁺ and Zn²⁺); b) form droplets from the mixture generated in a); c) allow reverse transcription to occur in the mixture of a) prior to or after droplet formation in b); d) amplify any cDNA in said droplet of b) or c); e) detect for the presence of cDNA; wherein reverse transcriptase is present in the sample if cDNA is detected in e) and the absence of cDNA in e) relates to an absence of reverse transcriptase. In one embodiment, the sample has been exposed to conditions effective to lyse viral particles. In one embodiment, one or more probes are added to the mixture of a). In one embodiment, the probe is labeled, for example, a fluorescent label. In one embodiment, the primer comprises a forward and a reverse primer. In one embodiment, the reverse primer is incubated with the template RNA prior to addition to the mixture of a). In one embodiment, an RNAse inhibitor is added to the mixture of a). In one embodiment, the amplification in d) comprises a polymerase chain reaction. Another embodiment further comprising quantifying reverse transcriptase enzyme or viral production present in said sample from the cDNA present in e).

One embodiment provides a method to screen for a reverse transcriptase inhibitor comprising: a) prepare a mixture of template RNA, nucleotides (deoxy triphosphates), reverse transcriptase, primer, DNA polymerase, and a possible reverse transcriptase inhibitor agent; b) form droplets from the mixture generated in a); c) allow reverse transcription to occur in the mixture of a) prior to or after droplet formation in b); d) amplify any cDNA in said droplet of b) or c); e) detect the presence of cDNA in d); if no cDNA is detected in e), then said agent is a reverse transcriptase inhibitor. In one embodiment, one or more probes are added to the mixture of a). In one embodiment, the probe is labeled. In one embodiment, the label fluorescent. In one embodiment, the primer comprises a forward and a reverse primer. In another embodiment, the reverse primer is incubated with the template RNA prior to addition to the mixture of a). In one embodiment, an RNAse inhibitor is added to the mixture of a). In another embodiment, the amplification in d) comprises a polymerase chain reaction.

One embodiment provides a method to screen for a reverse transcriptase (RT) with resistance to an inhibitor comprising: a) prepare a mixture of template RNA, nucleotides, reverse transcriptase, primer, DNA polymerase, and a reverse transcriptase inhibitor; b) form droplets from the mixture generated in a); c) allow reverse transcription to occur in the mixture of a) prior to or after droplet formation in b); d) amplify any cDNA in said droplet of b) or c); and e) detect the presence of cDNA in d); if cDNA is detected in e), then said reverse transcriptase is resistant to said inhibitor. Such inhibitors can include Nucleoside analog reverse-transcriptase inhibitors (NARTIs or NRTIs; including, Zidovudine, Didanosine, Zalcitabine, Stavudine, Lamivudine, Abacavir, Emtricitabine, and Entecavir,), nucleotide analog reverse-transcriptase inhibitors (NtARTIs or NtRTIs; including Tenofovir and Adefovir) and non-nucleoside reverse-transcriptase inhibitors (NNRTIs; including Efavirenz, Nevirapine, Delavirdine, Etravirine and Rilpivirine). This method allows for the detection of low abundant resistant RT (resistant to treatment with RT inhibitors) even in the presence of non-resistant RT (one could quantify the amount of resistance present in, for example, an HIV patient and stratify patients/determine resistance sooner than with conventional methods).

The present invention provides an improvement over prior technology in that ddPERT only requires that each individual RT enzyme is able to complete a single cDNA product through the length of the designed detection amplicon, while standard PERT or other RT enzyme assays require bulk processivity of RT. Additionally, the RT phase is allowed to go to completion.

In one embodiment, ddPERT quantifies absolutely those RT enzymes present in a reaction that are capable of generating cDNA (those that are active). Absolute method to measure/quantitate RTs at single level/one at a time. Can further use the methods described herein to characterize properties of the RT, such as ability to bind template, make cDNA (single or multiple cDNAs).

In one embodiment, ddPERT can distinguish between conditions that make more individual RT molecules active versus the condition (such as time, temperature, and buffer components) that promote the generation of multiple cDNAs from the same RT molecule.

In one embodiment, ddPERT is useful for detecting and determining more than one virus present in a sample.

In one embodiment, ddPERT is useful for quantifying viral vectors.

In one embodiment, ddPERT is useful for quantifying viral production.

In one embodiment, RT inhibitors can be used to test for drug resistant viruses.

In one embodiment, the present invention provides for the detection of RTs that are resistant to RT inhibitors in mixed RT populations such as clinical specimens.

The present application provides a method for the detection of reverse transcriptase utilizing droplet digital PCR in order to provide relative or absolute quantification of individual RT enzymes. Additional adaptations of the assay allow for determining RT characteristics, such as rate of cDNA synthesis and length of cDNA created.

Based on the disclosure provided herein, it can be seen that the assay and methods of the invention are less susceptible to bias at the RT reaction than prior techniques used in the art.

Another advantage of the present invention is that contrary to techniques such as ddPCR, the present technology does not require genomic sequence information. In one aspect, this allows for detecting unknown and potentially pathogenic retroviruses.

The present invention further provides kits for use with instruments such as those used herein from Bio-Rad, but also with other types of instruments where this technique is applicable. The kit is beneficial in terms of increased sensitivity, accuracy, speed and/or cost relative to alternative methods that rely on viral antigen or genome detection.

In one embodiment, ddPERT is useful for process control for industries that make vaccines, recombinant proteins, antibodies or other medical products in mammalian systems that can be contaminated by endogenous or exogenous retroviruses.

The present assay has useful clinical applications, including, but not limited to, detection of viral load from viruses such as Human Immunodeficiency Virus (HIV) and Human T-cell Lymphotropic Virus (HTLV). As disclosed herein, the ddPERT assay is sensitive to any RT enzyme from either known or unknown retroviruses, unlike genome- or antigen-based detection methods that require prior characterization of the viral target. In one embodiment, the present assay and methods are useful for routine screening of retroviral pathogens in blood products and/or transplant tissues. In one aspect, the method can be modified to confer specificity for detection of specific viral targets.

The assay of the present invention is useful in drug discovery as it can be used to monitor the production of retroviruses from model systems such as cell culture.

In one embodiment, the assay and methods can be used to screen chemical inhibitors of processes, such as retrovirus production, including direct RT inhibitors, as well as molecules that affect any other aspect of viral replication, packaging or export.

In one embodiment, ddPERT is useful as a sensitive drug screening method of compound libraries against purified RT enzymes. In particular ddPCR techniques are able to detect changes as low as 10% which would be useful for finding inhibitors that may have only slight effects that can be later optimized through typical pharmacological methods. The very minimal sample input required for ddPERT detection enables frequent sampling of culture supernatants over time when monitoring retroviral production in a model system.

The assay and methods of the invention can be used for diagnostics and can be used as part of a personalized medicine approach for developing and modifying treatments for a subject diagnosed with a disease, disorder, or condition.

Various aspects and embodiments of the invention are described in further detail below.

### BRIEF DESCRIPTION OF THE DRAWINGS

FIG. 1: Overview of droplet digital PCR.

FIGS. 2A-2C: PERT and ddPERT assays. Panel A illustrates the PERT assay. Reverse transcriptase (RT) makes cDNA from RNA template. For clarity, the many copies of MS2 primer and probe present are not shown. This cDNA template is then amplified by polymerase (P) while allowing for simultaneous detection of amplification. Primers are shown as gray arrows. Hydrolysis probes are shown at bottom right. Subsequent panels depict the release of fluorophore from quenchers as making a droplet positive. Panel B demonstrates a ddPCR variant (ddPERT) where the reaction is immediately partitioned into droplets which gives a more absolute count since only RT-containing droplets will be eventually read as positive. Panel C demonstrates a high sensitivity ddPERT variant where RT activity is allowed to proceed prior to droplet generation in which case either RT or a generated cDNA can segregate and will result in a positive droplet. Panel B and C would be taking place in roughly 20,000 droplets, not the four shown for demonstration. Each droplet contains all necessary components for reverse transcription, PCR amplification and detection of PCR amplification.

FIGS. 3A-3B: Comparison of ddPERT to standard PERT for duplicate standards. Result for M-MuLV is shown in (A) and AMV is shown in (B). On the left for each panel is the average number of positives in the total reaction calculated by Bio-Rad ddPCR software. On the right for each panel is the raw data for duplicate samples taken from the cfx384 thermocycler. Labelled arrows indicate what is being defined as the useful range for traditional PERT. Usable ranges of the assay for each standard are indicated by labelled arrows.

FIG. 4: Time course of 625 nanoUnits of M-MuLV recombinant standard. Duplicate reactions containing the same input were incubated at 42° C. at times stated prior to droplet generation. Additional RT incubation did not take place inside the droplet prior to heating to 95° C. to activate the polymerase, which explains the low amount present at 0 minutes. By contrast the absolute sample was incubate at 42 degrees Celsius for 60 minutes after droplet generation. The extreme variability of the pre-droplet incubated sample signal over time demonstrates the amount of amplification that can occur at this phase, thereby preventing typical PERT from being absolutely quantitative.

FIG. 5: Redesigned PERT assay. Primers and probes are listed as ddPERT LR in Table 2. Reverse transcriptase (RT), Polymerase (P) MS2 RNA (blue line), primers (blue arrows). Hydrolysis probes with FAM labeling (blue) or HEX (green), Internal zen quencher (grey) Iowa black dark quencher (black). Fluorescence is released from the quencher by the actions of polymerase that hydrolyses the probe.

FIG. 6. Results of assay described in FIG. 5 for AMV and M-MuLV. Total positive for the 3310 assay are blue bars while red bars indicate signal from both 1011 and 3310 assay which indicate a long cDNA was created. Both were assayed with 200 nanoUnits per assay.

FIG. 7. Absolute ddPERT assay comparing 10 microUnits of each enzyme. Absolute ddPERT is only absolute in the sense it quantifies absolutely those RT enzymes present in a reaction that are capable of generating cDNA, those that are active. When comparing RTs from different sources or species the reaction components may need to be optimized to the point that the conditions do not prevent RT from acting. In this regard absolute ddPERT is the solution to its own problem, in that it is the only current method that can distinguish between conditions that make more individual RT molecules active versus those conditions (time, temperature, buffer components including choice and concentration of divalent cation manganese or magnesium, and other PCR and RT reaction enhancers such as Betaine and the like) that promote the generation of multiple cDNAs from the same RT molecule. No upward increase in signal was observed after a pre-droplet incubation ranging from 0-10 minutes for either M-MuLV RT or AMV RT when using the 3310 assay or 1011 assays locations (FIG. 8).

FIG. 8: Stability of absolute ddPERT assay signal with differing length of incubation at room temperature prior to droplet generation.

FIG. 9: Diagram of workflow as a general example, changes in multiple parameters are tolerated.

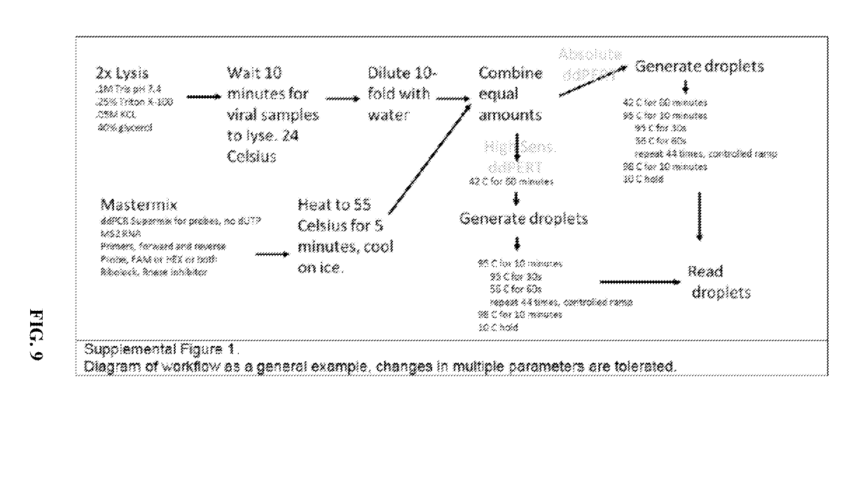

### DETAILED DESCRIPTION OF THE INVENTION

There is a need in the art for more accurate methods of detecting, identifying, and quantifying RT enzymes, viruses, etc. using techniques such as PCR. The present invention satisfies these needs.

The Product-Enhanced Reverse Transcriptase (PERT) assay is a quick and easy way to determine the presence of RT enzymes from a variety of sources. Herein is an enhancement of the sensitivity of this assay for applications such as the detection of very small amounts of endogenous RT from sources including unenriched primary patient material.

To do so, an existing PERT assay that uses bacteriophage MS2 RNA as template was modified so that it could be partitioned into a droplet digital PCR reaction for greater resolution. Each 20 microliter assay was partitioned into 20,000 nanoliter-sized droplets that contained template, primers, and a hotstart polymerase. The droplets were then thermocycled for 45 cycles and read using the Bio-Rad QX200 droplet reader. This novel method exhibited linear quantification of recombinant enzymes such as MuLV at similar dilutions to standard PERT samples that were run in parallel triplicate 10 microliter reactions. Modified cycling conditions were also shown to greatly enhance signal when a yes or no answer was all that was required, without introducing substantial false positive rates.

Droplet Digital PCR (ddPCR) is designed to work on the scale that single molecules of DNA per droplet are sufficient to promote amplification and detection of the amplification in each droplet. In contrast to standard PERT or other RT enzyme assays that may require bulk processivity of RT, ddPERT only requires that each individual RT enzyme is able to complete a single cDNA product through the length of the designed detection amplicon. Because of this low threshold it is theorized that virtually any RT enzyme will be detectable by this assay without the need to modify cation or buffer concentrations. The background for the assay is very low leading to accurate quantification at low concentrations. The lowest concentration measurable is only limited by inadequate sampling of the input material. This sensitivity allows for greater dilution of the sample which may effectively diminish the presence of inhibitors. The fact that each RT enzyme is interrogated singly makes this assay amenable to measuring other characteristics of the enzyme in addition to quantity present, which may be applicable to mixed populations of enzymes that are the result of mutation. In summary, demonstrated herein are increased sensitivity and other characteristics of ddPERT relative to standard PERT that will provide new practical applications of the assay.

### ABBREVIATIONS AND ACRONYMS

C-PERT—conventional PERT

cDNA—complementary DNA

dsDNA—double-stranded DNA

dd—droplet digital

ddPCR—droplet digital PCR

ddPERT—droplet digital

F-PERT—fluorescent PERT

HIV—Human Immunodeficiency Virus

HTLV—Human T-cell Lymphotropic Virus

M-MuLV—Moloney-Murine leukemia virus

mRNA—messenger RNA

PCR—polymerase chain reaction

PERT—product enhanced reverse transcriptase

RT—reverse transcriptase

RT-PCR—Reverse transcriptase PCR

SG-PERT—Sybr green PERT

STF-PERT—single tube fluorescent PERT

TM-PERT—TaqMan fluorogenic 5′-nuclease Product-Enhanced Reverse Transcriptase

### DEFINITIONS

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only; they do not exclude other defined values or other values within defined ranges for the radicals and substituents.

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%.

The term “biological sample,” as used herein, refers to samples obtained from a subject, including, but not limited to, skin, hair, tissue, blood, plasma, cells, sweat and urine. Further, laboratory samples can used as well, such as cell cultures, cell culture medium (e.g., that was used to allow the growth of human derived cell lines or primary human material (or non-human cells/material), vectors/plasmids.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “computer-readable medium” is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “fragment” or “segment” is a portion of an amino acid sequence, comprising at least one amino acid, or a portion of a nucleic acid sequence comprising at least one nucleotide.

The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the) XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

As used herein, an “instructional material” includes a publication, a recording, a diagram, or any other medium of expression which can be used to communicate the usefulness of the invention in the kit. The instructional material of the kit of the invention may, for example, be affixed to a container or be shipped together with a container. Alternatively, the instructional material may be shipped separately from the container with the intention that the instructional material.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “measuring the level of expression” or “determining the level of expression” as used herein refers to any measure or assay which can be used to correlate the results of the assay with the level of expression of a gene or protein of interest. Such assays include measuring the level of mRNA, protein levels, cDNA etc. and can be performed by assays such as northern and western blot analyses, binding assays, immunoblots, PCR etc.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “peptide” typically refers to short polypeptides.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “providing a prognosis” refers to providing information regarding the impact of the presence of for example a virus (e.g., as determined by the diagnostic methods of the present invention) on a subject's future health (e.g., expected morbidity or mortality).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A “recombinant polypeptide” is one which is produced upon expression of a recombinant polynucleotide.

The term “regulate” refers to either stimulating or inhibiting a function or activity of interest.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture. Sample can also include a composition generated in a laboratory, such as a composition comprising, for example, a vector that expresses a reverse transcriptase.

Reverse transcriptases are generally found in retroviruses, such as viruses described as follows: Genus Alpharetrovirus; type species: Avian leukosis virus; others include Rous sarcoma virus; Genus Betaretrovirus; type species: Mouse mammary tumour virus; Genus Gammaretrovirus; type species: Murine leukemia virus; others include Feline leukemia virus; Genus Deltaretrovirus; type species: Bovine leukemia virus; others include the cancer-causing Human T-lymphotropic virus; Genus Epsilonretrovirus; type species: Walleye dermal sarcoma virus; Genus Lentivirus; type species: Human immunodeficiency virus 1; others include Simian, Feline immunodeficiency viruses; Genus Spumavirus; type species: Simian foamy virus, as well as metaviridae, Pseudoviridae, retroviridae (e.g., HIV), family Hepadnaviridae (e.g., Hepatitis B virus); family Caulimoviridae (e.g. Cauliflower mosaic virus); and endogenous retrovirus.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2×standard saline citrate (SSC), 0.1% SDS at 50° C.; preferably in 7% (SDS), 0.5 M 25 NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide or nucleic acid which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

The term to “treat,” as used herein, means reducing the frequency with which symptoms are experienced by a patient or subject or administering an agent or compound to reduce the frequency with which symptoms are experienced.

The terms “comprises,” “comprising,” and the like can have the meaning ascribed to them in U.S. Patent Law and can mean “includes,” “including” and the like. As used herein, “including” or “includes” or the like means including, without limitation.

### ASPECTS OF THE INVENTION

The present invention can be practiced with a system such as the Bio-Rad system as exemplified herein as well as other digital PCR systems that are available. For example, the invention can be used with digital PCR systems and equipment from RainDance Technologies. Additionally, the invention can be used with digital PCR systems and equipment from Life Technologies (now ThermoFisher). The present methods can be used or adapted for use with plates/wells. In some cases minor modifications may be necessary to practice the invention using other systems, but the adaptions will be primarily optimization.

In other embodiments, the present invention can also be practiced with systems not yet available if the general procedures and methods of the invention can be used with them or take advantage of the properties of new systems that become available, as long as the results and efficiencies obtained are encompassed by or similar to those disclosed herein.

### PERT

The PERT assay is an assay for the detection of reverse transcriptase (RT) activity and has been reported to be up more sensitive than conventional RT assays. The assay is an RT dependent polymerase chain reaction (PCR) and therefore combines the broad specificity of conventional RT assays with the sensitivity of PCR. Like conventional RT assays, it is used to detect RT activity packaged into extracellular retrovirus particles. The assay involves converting an RNA template (generally a known amount and sequence of RNA) to cDNA and amplifying the cDNA using product specific primers (thus the reaction mixture will contain sample, RNA template, primers, (For RT to generate cDNA and optionally for amplification of the cDNA by PCR for detection), nucleotides and generally a buffer and an RNAse inhibitor). Since no exogenous RT activity is added, cDNA will only be generated if the sample itself contains RT activity. If no RT activity is present, no product will be detected. In one embodiment, the TaqMan® technology can be used with the real time detection of specific PERT reaction product.

ddPCR/Droplet Generation

The present disclosure includes compositions, methods, and kits for manipulation of samples in droplets, e.g., using droplet digital PCR. The droplets described herein can include droplets generated by devices described in International Application Publication No. WO/2010/036352, which is hereby incorporated by reference in its entirety. One or more enzymatic reactions can occur in a droplet.

In Digital Droplet PCR (ddPCR) the PCR solution is divided into smaller reactions through for example a water oil emulsion technique, which are then made to run PCR individually. The PCR sample can be partitioned into nanoliter-size samples and encapsulated into oil droplets. The oil droplets are made using a droplet generator that applies a vacuum to each of the wells. Approximately 20,000 oil droplets are made from each 20 μL sample.

Splitting a sample into small reaction volumes as described herein can enable the use of reduced amounts of reagents, thereby lowering the material cost of the analysis. Reducing sample complexity by partitioning can also improve the dynamic range of detection, since higher-abundance molecules can be separated from low-abundance molecules in different compartments, thereby allowing lower-abundance molecules greater proportional access to reaction reagents, which in turn can enhance the detection of lower-abundance molecules.

Droplets can be generated having an average diameter of about, more than about, less than about, or at least about 0.001, 0.01, 0.05, 0.1, 1, 5, 10, 20, 30, 40, 50, 60, 70, 80, 100, 120, 130, 140, 150, 160, 180, 200, 300, 400, or 500 microns. The average diameter of the droplets can be about 0.001 microns to about 0.01 microns, about 0.001 microns to about 0.005 microns, about 0.001 microns to about 0.1 microns, about 0.001 microns to about 1 micron, about 0.001 microns to about 10 microns, about 0.001 microns to about 100 microns, about 0.001 microns to about 500 microns, about 0.01 microns to about 0.1 microns, about 0.01 microns to about 1 micron, about 0.01 microns to about 10 microns, about 0.01 microns to about 100 microns, about 0.01 microns to about 500 microns, about 0.1 microns to about 1 micron, about 0.1 microns to about 10 microns, about 0.1 microns to about 100 microns, about 0.1 microns to about 500 microns, about 1 micron to about 10 microns, about 1 micron to about 100 microns, 1 micron to about 500 microns, about 10 microns to about 100 microns, about 10 microns to about 500 microns, or about 100 microns to about 500 microns.

Droplet volume can be about, more than about, less than about, or at least about 0.001 nL, 0.01 nL, 0.1 nL, 1 nL, 10 nL, or 100 nL. Droplets can be generated using, e.g., RAINSTORM™ (RAINDANCE™), microfluidics from ADVACED LIQUID LOGIC, or ddPCR™ (BIO-RAD).

Microfluidic methods of producing emulsion droplets using microchannel cross-flow focusing or physical agitation can produce either monodisperse or polydisperse emulsions. The droplets can be monodisperse droplets. The droplets can be generated such that the size of said droplets does not vary by more than plus or minus 5% of the average size of said droplets. The droplets can be generated such that the size of said droplets does not vary by more than plus or minus 2% of the average size of said droplets. A droplet generator can generate a population of droplets from a single sample, wherein none of the droplets can vary in size by more than plus or minus 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

Both the flow rate in a droplet generator and the length of nucleic acids in a sample can have an impact on droplet generation. One way to decrease extension is to decrease flow rate; however, this can have the undesirable side effect of lower throughput and also increased droplet size. Long nucleic acids can disrupt droplet formation in extreme cases, resulting in a steady flow rather than discrete droplets. Reducing nucleic acid size in a sample can improve droplet formation when nucleic acid loads are high. Samples with high nucleic acid loads (e.g., high DNA loads, high RNA loads, etc.) can be used. Reducing the length of nucleic acids in a sample (e.g., by digestion, sonication, heat treatment, or shearing) can improve droplet formation.

Higher mechanical stability can be useful for microfluidic manipulations and higher-shear fluidic processing (e.g., in microfluidic capillaries or through 90 degree turns, such as valves, in a fluidic path). Pre- and post-thermally treated droplets or capsules can be mechanically stable to standard pipette manipulations and centrifugation.

A droplet can be formed by flowing an oil phase through an aqueous sample. A droplet can comprise a buffered solution and reagents for performing an amplification reaction, e.g., a PCR reaction, including nucleotides, primers, probe(s) for fluorescent detection, template nucleic acids, DNA polymerase enzyme, and/or reverse transcriptase enzyme.

A droplet can comprise a buffered solution and reagents for performing an enzymatic reaction (e.g., a PCR). The buffered solution can comprise about, more than about, at least about, or less than about 1, 5, 10, 15, 20, 30, 50, 100, or 200 mM Tris. A droplet can comprise one or more buffers including, e.g., TAPS, bicine, Tris, Tricine, TAPSO, HEPES, TES, MOPS, PIPES, cacodylate, SSC, ADA, ACES, cholamine chloride, acetamidoglycine, glycinamide, maleate, phosphate, CABS, piperidine, glycine, citrate, glycylglycine, malate, formate, succinate, acetate, propionate, pyridine, piperazine, histidine, bis-tris, ethanolamine, carbonate, MOPSO, imidazole, BIS-TRIS propane, BES, MOBS, triethanolamine (TEA), HEPPSO, POPSO, hydrazine, Trizma (tris), EPPS, HEPPS, bicine, HEPBS, AMPSO, taurine (AES), borate, CHES, 2-amino-2-methyl-1-propanol (AMP), ammonium hydroxide, methylamine, or MES. The pH of the droplet can be about 2, 2.5, 3, 3.5, 4, 4.5, 5, 5.5, 6, 6.5, 6.6, 6.7, 6.8, 6.9, 7, 7.1, 7.2, 7.3, 7.4, 7.5, 7.5, 7.6, 7.7, 7.8, 7.9, 8, 8.1, 8.2, 8.3, 8.4, 8.5, 8.6, 8.7, 8.8, 8.9, 9, 9.5, 10, 10.5, 11, 11.5, 12, or 12.5. The pH of the droplet can be about 5 to about 9, about 5 to about 8, about 5 to about 7, about 6.5 to about 8, about 6.5 to about 7.5, about 6 to about 7, about 6 to about 9, or about 6 to about 8.

A droplet can comprise a salt, e.g., potassium acetate, potassium chloride, magnesium acetate, magnesium chloride, sodium acetate, or sodium chloride. The concentration of potassium chloride can be about, more than about, at least about, or less than about 10, 20, 30, 40, 50, 60, 80, 100, 200 mM. The buffered solution can comprise about 15 mM Tris and about 50 mM KCl.

A droplet can comprise nucleotides. The nucleotides can comprise deoxyribonucleotide triphosphate molecules, including dATP, dCTP, dGTP, dTTP, in concentrations of about, more than about, less than about, or at least about 50, 100, 200, 300, 400, 500, 600, or 700 .mu.M each. dUTP can be added within a partition, e.g., an aqueous phase of an emulsion, to a concentration of about, less than about, more than about, or at least about 50, 100, 200, 300, 400, 500, 600, 700, 800, 900, or 1000 .mu.M. The ratio of dUTP to dTTP in a droplet can be about 1:1000, 1:500, 1:250, 1:100, 1:75, 1:50, 1:40, 1:30, 1:20, 1:10, 1:5, 1:4, 1:3, 1:2, or 1:1.

A droplet can comprise one or more divalent cations. The one or more divalent cations can be, e.g., Mg²⁺, Mn²⁺, Cu²⁺, Co²⁺, or Zn²⁺. Magnesium chloride (MgCl₂) can be added to a droplet at a concentration of about, more than about, at least about, or less than about 1.0, 2.0, 3.0, 4.0, or 5.0 mM. The concentration of MgCl₂ can be about 3.2 mM. Magnesium sulfate can be substituted for magnesium chloride, at similar concentrations. A droplet can comprise both magnesium chloride and magnesium sulfate. A wide range of common, commercial PCR buffers from varied vendors can be substituted for the buffered solution.

A non-ionic Ethylene Oxide/Propylene Oxide block copolymer can be added to a droplet in a concentration of about, more than about, less than about, or at least about 0.1%, 0.2%, 0.3%, 0.4%, 0.5%, 0.6%, 0.7%, 0.8%, 0.9%, or 1.0%. A droplet can comprise a biosurfactant. Common biosurfactants include non-ionic surfactants such as Pluronic F-68, Tetronics, Zonyl FSN. Pluronic F-68 can be present at a concentration of about 0.5% w/v.

### Additives

A droplet can comprise one or more additives including, but not limited to, non-specific background/blocking nucleic acids (e.g., salmon sperm DNA), biopreservatives (e.g., sodium azide), PCR enhancers (e.g., betaine (N,N,N-trimethylglycine; [carboxymethyl]trimethylammonium), trehalose, etc.), and/or inhibitors (e.g., RNAse inhibitors). A GC-rich additive comprising, e.g., betaine and DMSO, can be added to samples assayed in the methods provided herein.

The one or more additives can include a non-specific blocking agent such as BSA or gelatin from bovine skin. The gelatin or BSA can be present in a concentration range of approximately 0.1 to about 0.9% w/v. Other blocking agents can include betalactoglobulin, casein, dry milk, or other common blocking agents. In some cases, the concentration of BSA and gelatin are about 0.1% w/v.

The one or more additives can include 2-pyrrolidone, acetamide, N-methylpyrolidone (NMP), B-hydroxyethylpyrrolidone (HEP), propionamide, NN-dimethylacetamide (DMA), N-methylformamide (MMP), NN-dimethylformamide (DMF), formamide, N-methylacetamide (MMA), polyethylene glycol, tetramethylammonium chloride (TMAC), 7-deaza-2′-deoxyguanosine, T4 gene 32 protein, glycerol, or nonionic detergent (Triton X-100, Tween 20, Nonidet P-40 (NP-40), Tween 40, SDS (e.g., about 0.1% SDS)), salmon sperm DNA, sodium azide, formamide, dithiothreitol (DTT), betamercaptoethanol (BME), 2-mercaptoethylamine-HCl, tris(2-carboxythyl)phosphine (TCEP), cysteine-HCl, or a plant polysaccharide. The one or more additives can be ethanol, ethylene glycol, dimethylacetamide, dimethylformamide, or suphalane.

### Primers

A droplet can comprise oligonucleotide primers. The oligonucleotide primers can be used for amplification. Primers for amplification within a droplet can have a concentration of about, more than about, less than about, or at least about 0.1, 0.2, 0.3, 0.4, 0.5, 0.6, 0.7, 0.8, 0.9, or 1.0 μM. The concentration of each primer can be about 0.5 μM. Primers can be designed according to known parameters for avoiding secondary structures and self-hybridization. Different primer pairs can anneal and melt at about the same temperatures, for example, within about 1, 2, 3, 4, 5, 6, 7, 8, 9 or 10° C. of another primer pair. In some cases, greater than about 1, 2, 3, 4, 5, 6, 7, 8, 9, 10, 15, 20, 25, 30, 35, 40, 45, 50, 100, 200, 500, 1000, 5000, 10,000 or more primers are initially used. Such primers may be able to hybridize to the genetic targets described herein. About 2 to about 10,000, about 2 to about 5,000, about 2 to about 2,500, about 2 to about 1,000, about 2 to about 500, about 2 to about 100, about 2 to about 50, about 2 to about 20, about 2 to about 10, or about 2 to about 6 primers can be used.

Primers can be prepared by a variety of methods including but not limited to cloning of appropriate sequences and direct chemical synthesis using methods well known in the art (Narang et al., Methods Enzymol. 68:90 (1979); Brown et al., Methods Enzymol. 68:109 (1979)). Primers can also be obtained from commercial sources such as Integrated DNA Technologies, Operon Technologies, Amersham Pharmacia Biotech, Sigma, or Life Technologies. The primers can have an identical melting temperature. The melting temperature of a primer can be about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 81, 82, 83, 84, or 85.degree. C. The melting temperature of a primer can be about 30 to about 85° C., about 30 to about 80° C., about 30 to about 75° C., about 30 to about 70° C., about 30 to about 65° C., about 30 to about 60° C., about 30 to about 55° C., about 30 to about 50° C., about 40 to about 85° C., about 40 to about 80° C., about 40 to about 75° C., about 40 to about 70° C., about 40 to about 65° C., about 40 to about 60° C., about 40 to about 55° C., about 40 to about 50° C., about 50 to about 85° C., about 50 to about 80° C., about 50 to about 75° C., about 50 to about 70° C., about 50 to about 65° C., about 50 to about 60° C., about 50 to about 55° C., about 52 to about 60° C., about 52 to about 58° C., about 52 to about 56° C., or about 52 to about 54° C.

The lengths of the primers can be extended or shortened at the 5′ end or the 3′ end to produce primers with desired melting temperatures. One of the primers of a primer pair can be longer than the other primer. The 3′ annealing lengths of the primers, within a primer pair, can differ. Also, the annealing position of each primer pair can be designed such that the sequence and length of the primer pairs yield the desired melting temperature. An equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The TM (melting or annealing temperature) of each primer can be calculated using software programs such as Net Primer (free web based program at premierbiosoft.com/netprimer/index.html). The annealing temperature of the primers can be recalculated and increased after any cycle of amplification, including but not limited to about cycle 1, 2, 3, 4, 5, about cycle 6 to about cycle 10, about cycle 10 to about cycle 15, about cycle 15 to about cycle 20, about cycle 20 to about cycle 25, about cycle 25 to about cycle 30, about cycle 30 to about cycle 35, or about cycle 35 to about cycle 40. After the initial cycles of amplification, the 5′ half of the primers can be incorporated into the products from each loci of interest; thus the TM can be recalculated based on both the sequences of the 5′ half and the 3′ half of each primer.

The annealing temperature of the primers can be recalculated and increased after any cycle of amplification, including but not limited to about cycle 1, 2, 3, 4, 5, about cycle 6 to about cycle 10, about cycle 10 to about cycle 15, about cycle 15 to about cycle 20, about cycle 20 to about cycle 25, about cycle 25 to about cycle 30, about cycle 30 to about 35, or about cycle 35 to about cycle 40. After the initial cycles of amplification, the 5′ half of the primers can be incorporated into the products from each loci of interest, thus the TM can be recalculated based on both the sequences of the 5′ half and the 3′ half of each primer.

### Polymerases

A droplet can comprise a polymerase. The polymerase can be a DNA polymerase. The DNA polymerase can be, e.g., T4 DNA polymerase, DEEP VENT™ DNA polymerase, LONGAMP® Tag, PHUSION® High Fidelity DNA polymerase, LONGAMP® Hot Start Taq, Crimson LONGAMP® Taq, Taq DNA polymerase, Crimson Taq DNA polymerase, ONETAQ® DNA polymerase, QUICK-LOAD® DNA polymerase, VENTR® DNA polymerase, Hemo KLENTAQ®, Bsu DNA polymerase, DNA polymerase I, DNA Polymerase I, Large (Klenow), Klenow Fragment, Phi29 DNA polymerase, Pfu DNA polymerase, Pfx DNA polymerase, Tth DNA polymerase, Vent DNA polymerase, bacteriophase 29, REDTAQ™, or T7 DNA polymerase. The DNA polymerase can comprise 3′ to 5′ exonuclease activity. The DNA polymerase can comprise 5′ to 3′ exonuclease activity. The DNA polymerase can comprise both 3′ to 5′ exonuclease activity and 5′ to 3′ exonuclease activity. The DNA polymerase can comprise neither 3′ to 5′ exonuclease activity nor 5′ to 3′ exonuclease activity. The DNA polymerase can comprise strand displacement activity. In some cases, the DNA polymerase does not comprise strand displacement activity.

A droplet can comprise a reverse transcriptase. The reverse transcriptase can be AMV reverse transcriptase or M-MuLV reverse transcriptase. The RNA polymerase can comprise 5′ to 3′ exonuclease activity. The reverse transcriptase can comprise both 3′ to 5′ exonuclease activity and 5′ to 3′ exonuclease activity. The reverse transcriptase can comprise neither 3′ to 5′ exonuclease activity nor 5′ to 3′ exonuclease activity. The reverse transcriptase can comprise strand displacement activity. In some embodiments, the reverse transcriptase does not comprise strand displacement activity.

A droplet can comprise an RNA polymerase. The RNA polymerase can be, e.g., phi6 RNA polymerase, SP6 RNA polymerase, or T7 RNA polymerase.

In some embodiments, a droplet comprises Poly(U) polymerase or Poly(A) polymerase.

### Amplification

Polynucleotides (e.g., cDNA) may be amplified. In some embodiments, polynucleotides are amplified while in a partition (e.g., aqueous phase of an emulsion, e.g., droplet).

In some embodiments, the amplification comprises polymerase chain reaction (PCR), digital PCR, reverse-transcription PCR, quantitative PCR, real-time PCR, isothermal amplification, linear amplification, or isothermal linear amplification, quantitative fluorescent PCR (QF-PCR), multiplex fluorescent PCR (MF-PCR), single cell PCR, restriction fragment length polymorphism PCR(PCR-RFLP), PCR-RFLP/RT-PCR-RFLP, hot start PCR, nested PCR, in situ polony PCR, in situ rolling circle amplification (RCA), bridge PCR (bPCR), picotiter PCR, digital PCR, droplet digital PCR, or emulsion PCR (emPCR). Other suitable amplification methods include ligase chain reaction (LCR (oligonucleotide ligase amplification (OLA)), transcription amplification, cycling probe technology (CPT), molecular inversion probe (MIP)PCR, self-sustained sequence replication, selective amplification of target polynucleotide sequences, consensus sequence primed polymerase chain reaction (CP-PCR), arbitrarily primed polymerase chain reaction (AP-PCR), transcription mediated amplification (TMA), degenerate oligonucleotide-primed PCR (DOP-PCR), multiple-displacement amplification (MDA), strand displacement amplification (SDA), and nucleic acid based sequence amplification (NABSA). Other amplification methods that can be used herein include those described in U.S. Pat. Nos. 5,242,794; 5,494,810; 4,988,617; and 6,582,938.

Mmultiple-displacement amplification (MDA) can be a non-PCR based amplification technique that can involve annealing multiple primers (e.g., hexamer primers) to a polynucleotide template, and initiating DNA synthesis (e.g., using Phi 29 polymerase). When DNA synthesis proceeds to the next synthesis starting site, the polymerase can displace the newly produced DNA strand and continues its strand elongation. Strand displacement can generate newly synthesized single stranded DNA template to which other primers can anneal. Further primer annealing and strand displacement on the newly synthesized template can result in a hyper-branched DNA network. The sequence debranching during amplification can result in a high yield of products. To separate the DNA branching network, one or more S1 nucleases can be used to cleave the fragments at displacement sites. The nicks on the resulting DNA fragments can be repaired by DNA polymerase I. The generated DNA fragments can be directly used for analysis or be ligated to generate genomic libraries for further sequencing analysis. MDA is described, e.g., in U.S. Pat. No. 7,074,600.

A hot start PCR can be performed wherein the reaction is heated to 95° C. for two minutes prior to addition of the polymerase or the polymerase can be kept inactive until the first heating step in cycle 1. Hot start PCR can be used to minimize nonspecific amplification. Other strategies for and aspects of amplification suitable for use in the methods described herein are described in U.S. Patent Application Publication No. 2010/0173394 A1, published Jul. 8, 2010, which is incorporated herein by reference.

Any number of PCR cycles can be used to amplify the DNA, e.g., about, more than about, at least about, or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 cycles. The number of amplification cycles can be about 1 to about 45, about 10 to about 45, about 20 to about 45, about 30 to about 45, about 35 to about 45, about 10 to about 40, about 10 to about 30, about 10 to about 25, about 10 to about 20, about 10 to about 15, about 20 to about 35, about 25 to about 35, about 30 to about 35, or about 35 to about 40.

Thermocycling reactions can be performed on samples contained in droplets.

### Computers

A computer can be used to store and process the data. A computer-executable logic can be employed. A computer can be useful for displaying, storing, retrieving, or calculating diagnostic results from the molecular profiling; displaying, storing, retrieving, or calculating raw data from genomic or nucleic acid expression analysis; or displaying, storing, retrieving, or calculating any sample or patient information useful in the methods described herein. Provided herein are systems comprising computer readable instructions for performing methods described herein. Provided herein are computer readable medium comprising instructions which, when executed by a computer, cause the computer to perform methods described herein.

### Kits

Provided herein are kits for performing methods described herein. The kits can comprise one or more restriction enzymes, endonucleases, exonucleases, ligases, polymerases, RNA polymerases, DNA polymerases, reverse transcriptases, topoisomerases, kinases, phosphatases, buffers, salts, metal ions, reducing agents, BSA, spermine, spermidine, glycerol, oligonucleotides, primers, probes, or labels (e.g., fluorescent labels). The kits can comprises one or more sets of instructions.

### Applications of the Technology

ddPERT is useful to quantify viral vectors or viral production from model systems. Designing a kit for use with the, for example, Bio-Rad system is encompassed by the invention, as will adaptation for other systems. The present invention is useful in terms of increased sensitivity, accuracy, speed and/or cost versus alternative methods that rely on viral antigen or genome detection.

One aspect ddPERT provides is in process control for industries that make vaccines, recombinant proteins, antibodies, or other medical products in mammalian systems that can be contaminated by endogenous or exogenous retroviruses. High sensitivity ddPERT would be ideal for this application.

This assay has clinical application in the detection of viral load from viruses such as Human Immunodeficiency Virus (HIV) and HTLV. The ddPERT assay of the invention is sensitive to any RT enzyme from either known or unknown retroviruses, unlike genome- or antigen-based detection methods that require prior characterization of the viral target. Thus, there is utility for this assay in routine screening of retroviral pathogens in blood products and/or transplant tissues. It is possible that future modifications of the reaction conditions will confer specificity for detection of specific viral targets.

This assay has application in drug discovery as it can be used to monitor the production of retroviruses from model systems such as cell culture. As such, it can be used to screen chemical inhibitors of this process, including direct RT inhibitors, as well as molecules that affect any other aspect of viral replication, packaging or export. ddPERT will enable sensitive drug screening of compound libraries against purified RT enzymes. In particular ddPCR techniques are able to detect changes as a low as 10% which would be useful for finding inhibitors that may have only slight effects that can be later optimized through typical pharmacological methods. The present invention provides further improvements over the methods of the prior art, for example, the very minimal sample input required for ddPERT detection enables frequent sampling of culture supernatants over time when monitoring retroviral production in a model system.

A trend was noticed while using probe assays for the detection of mutations that if a probe is not an exact match to the DNA sequence the resulting positive droplet is intermediate in intensity when compared to the wild-type sequence. This knowledge can be used to determine the fidelity of an RT enzyme by counting the fraction of droplets that are of normal and intermediate intensities. It may not be possible to determine an absolute rate of mutation, giving that different mutations affect probe hybridization differently, however it is likely that a relative rate of mutation introduced by RT could be easily interpreted. Since ddPCR has two channels for detection, two probes can be used simultaneously to increase the amount of sequence that is interrogated in each positive droplet. Such a two probe assay was designed.

TABLE 1

| | | | |
|---|---|---|---|
| reverse primer 2 | Gccttagcagtgccctgtct | | published |
| | (SEQ ID NO: 16) | | TM-PERT |
| forward primer 2 | Aacatgctcgagggcctta | | published |
| | (SEQ ID NO: 17) | | TM-PERT |
| probe 2 | Cagtgggatgctcctacatgtc | hex zen iowablack | self |
| | (SEQ ID NO: 18) | | |
| probe 3 | Aagcgttgacgctccctacg | fam zen iowablack | self |
| | (SEQ ID NO: 19) | | |

A clinical extension of this assay would be the addition of currently used RT inhibitors such as AZT (azidothymidine, Ziduvidine) to the RT reaction. When used in conjunction with absolute ddPERT assay in side-by-side comparisons of the same patient sample with and without inhibitor present, an absolute fractional abundance of resistant versus sensitive RT enzymes present will be determined, which could provide an early indication of treatment failure in that patient. It is of note that unlike traditional PERT where RT inhibitors could potentially diminish the signal created by RT or amplified effects by acting on polymerase, ddPCR methods are much less susceptible as droplets with lower positive signal still give accurate quantification provided they are distinguishable from negative.

It is conventional to study the aspects of individual RT enzymes through molecular cloning and recombinant protein expression. This is tedious, requires additional equipment and expertise for purification. In general, large amounts of the enzyme most be produced to study. For example they would measure the protein concentration and determine how many enzymes are present based on the molecular weight of the enzyme and then conduct tests of processivity, fidelity and the like.

The assays described herein for fidelity, rate of cDNA creation and cDNA length are able to be conducted on relatively small numbers of enzymes, hundreds or thousands of molecules or higher. This allows these aspects to be tested in native viruses from patient samples or natural sources, or possibly after viral expansion in cell lines. This allows a more native presentation of RT in comparison to bacterial expression systems that produce recombinant enzyme that may also be epitope tagged to facilitate purification, both of which could impact protein folding and/or catalytic activity.

In one embodiment, the methods of the invention can be used to study RT characteristics from cell culture models whereby you are able to mutagenize the RT sequence. As such this may be a convenient and high throughput screening platform for the generation of recombinant RT with characteristics desirable for molecular biology such as the ability to complete full-length cDNAs. In another embodiment, plasmids containing a cDNA sequence for RT may be utilized within an in vitro transcription and translation mixture to generate small amounts of RT, which are amenable to detection by the highly sensitive ddPERT assay. This would enable rapid screening methods that utilize varying plasmid constructs with altered RT sequences that utilize random mutagenesis cassettes to create tremendous diversity.

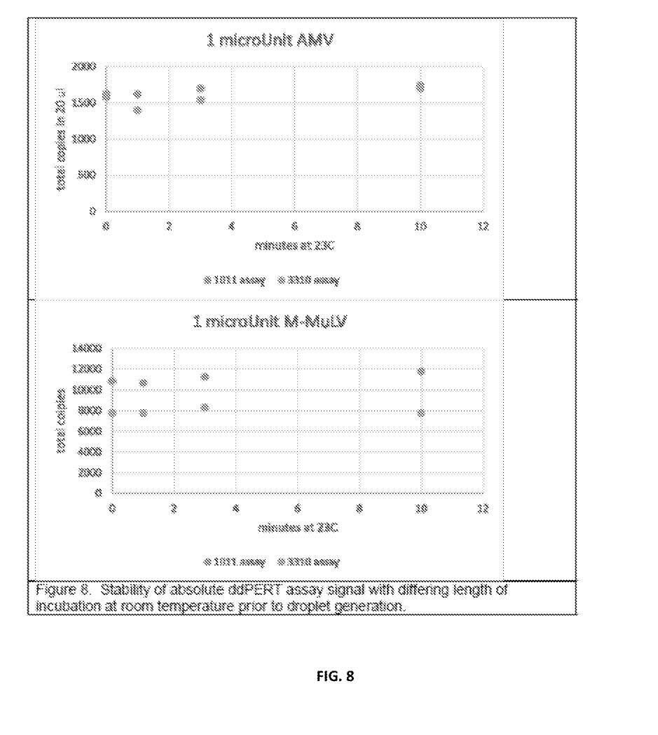

Differences in absolute counts were observed for the M-MuLV RT when using 3310 or 1011 assays, but this is not so for AMV RT (FIG. 8). Since this is stable for the timer period tested, it is not likely to be related to the creation of cDNAs that dissociate from RT and create additional positive droplets. This is to be related to differences in the secondary structure of the MS2 RNA at these locations and that this represents differential capacity of the AMV and M-MuLV to overcome this challenge. These and additional assays designed to the MS2 genome or other RNA sources provides a convenient test to screen for RTs that can overcome these challenges, that is RNA secondary structure that prevents the elongation of cDNAs. This would include the use of model systems that allow for the editing or other alteration of RNA genomes or RNA transcripts in order to introduce particular sequences or secondary structure.

There are additional technologies that perform digital PCR such as the RainDance Technologies' RainStorm digital droplet which partitions into 1 million droplets and the Thermo-Fisher Scientific chip-based partitioning system Quantstudio that has roughly 20,000 partitions. These and others are a suitable platform for use of these assays.

The following example is intended to further illustrate certain embodiments of the invention and are not intended to limit the scope of the invention in any way.

### EXAMPLE

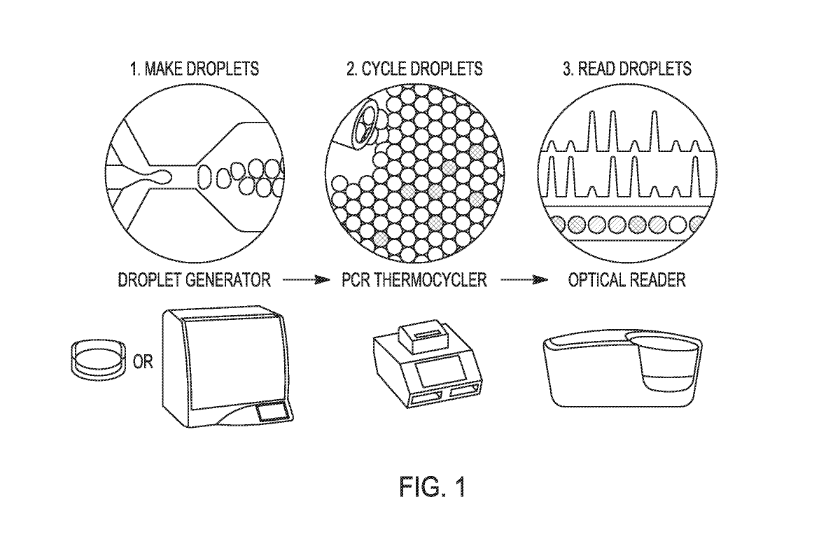

A significant recent advancement in PCR technology is the advent of digital PCR, wherein the PCR reaction can be fractionated in some way so that each partition is read in a binary manner as to whether or not target DNA is present. This allows for the absolute counting of the presence of individual DNA species. One such technology is droplet digital PCR from Bio-Rad. Utilizing Bio-Rad equipment, individual PCR reactions were split into 20,000 nanoliter droplets, cycled to endpoint, and then determined the presence of fluorescence in each droplet (FIG. 1). The oil composition, buffer composition, microfluidic plates and reader technology are the subject of multiple patents by Bio-Rad. Reaction master mixes that provide all necessary components for PCR were ordered from Bio-Rad, specifically ddPCR supermix for probes (no dUTP) part number 186-30224.

The present invention utilizes in part the combination of two technologies. Following optimization and use of the presently disclosed technology (referred to as ddPERT herein), the data demonstrate that ddPERT is capable of the absolute quantification of the presence of RT enzymes. Although ddPCR is known to provide absolute counts of DNA molecules and PERT is known to detect the presence of RT, the procedures described herein that provide in part the combination of the two has never been taught or contemplated in the art, nor was the unexpected result of the ability to determine absolute quantity of RT as presently demonstrated.

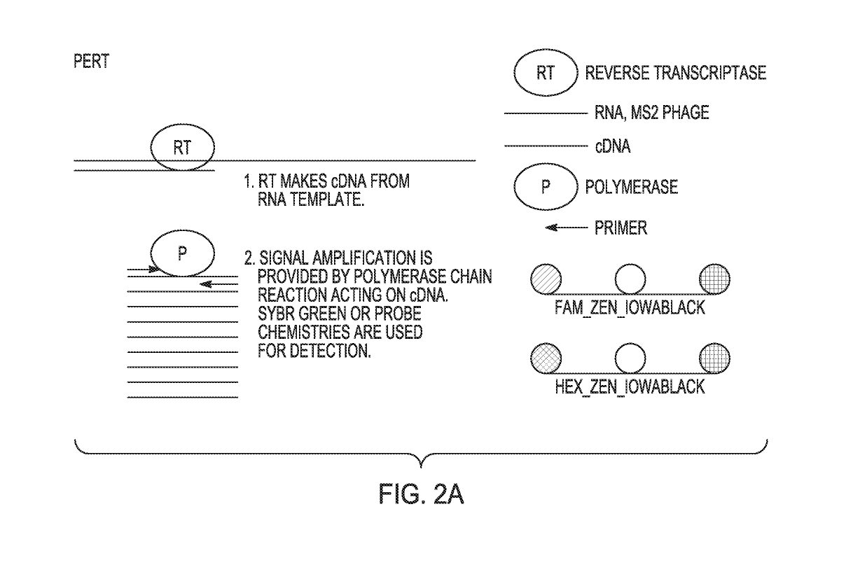

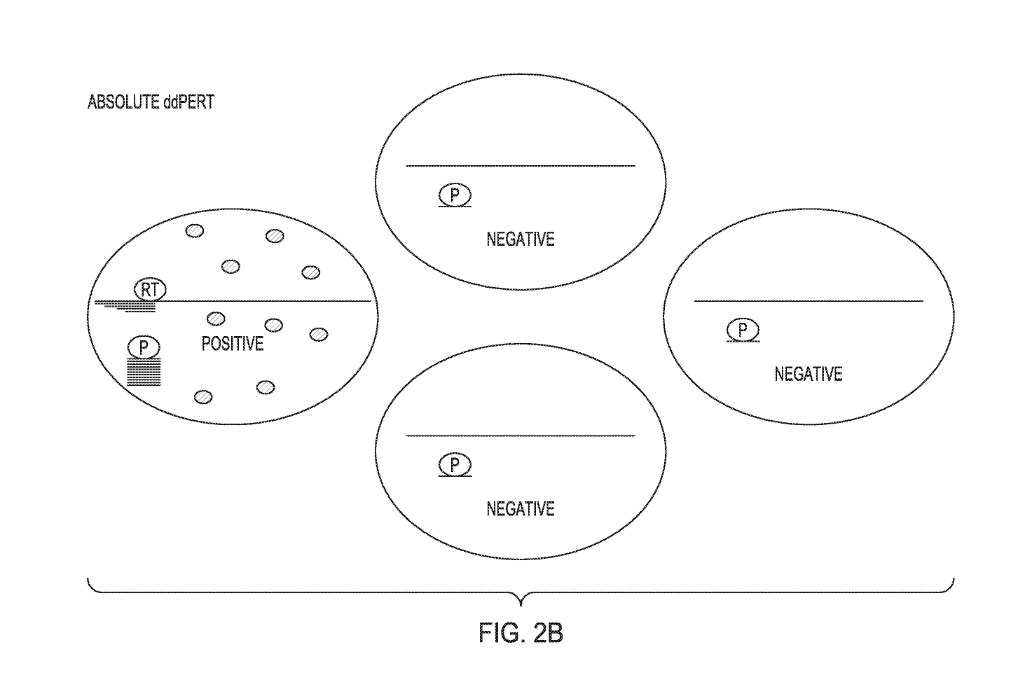

FIG. 2 shows a diagram of a typical PERT assay (panel A) and demonstrates the problems with current PERT assays in that they are not absolute counts of RT enzymes. This is so because it is possible for a single RT to make multiple cDNAs from multiple MS2 RNA templates. FIG. 3 shows an example using Moloney-Murine leukemia virus (M-MuLV) reverse transcriptase from New England Biolabs (part number M0253). The use of RTs from different viral or recombinant sources, different reaction conditions (mastermixes), and variation in the temperature or duration of this phase can introduce high amounts of variability in the number of cDNAs created by each individual RT; therefore, this does not allow for absolute amounts of RT to be established accurately. The immediate partitioning of this reaction into droplets results in only those droplets with RT enzyme being counted as RT positive and therefore gives an absolute count of RT. Allowing the RT reaction to take place prior to droplet generation (Panel C) results in either droplets with RT or a previously generated cDNA creating a positive droplet which results in an amplification of signal. This can be exploited to show the true presence of RT in applications where the presence or absence of RT is most important such as determining the presence of retroviral contamination into biological products.

In samples where cDNA or DNA contamination is suspected rather than RT activity, a high sensitivity assay with multiple incubation timespans for RT would be useful in eliminating false positives, as products of previous assay cannot be amplified by RT. This of course requires that polymerase be prevented from acting on DNA or cDNA via partitioning or hotstart modalities.

The first step in ddPERT is sample preparation. In order to assay RT from viral particles, these particles must be lysed to allow the release of RT into the reaction mixture. An example of a lysis solution is 0.1 M Tris pH 7.4, 0.25% Triton X-100, 0.05 M KCL, 40% glycerol in a 1:1 ratio to be used with sample material in RPMI to lyse the particles. After lysis, this mixture was diluted further 9:1 with water prior to droplet generation. This dilution is a standard procedure for performing PERT. At this 10-fold dilution no inhibition of droplet generation was found and it was determined that the PCR components were still functional.

Reactions were designed to be 1:1 combinations of diluted RT and all other necessary reaction components. This includes 10.3 microliter of supermix (2×, Bio-Rad), 0.1 microliter of MS2 phage RNA (0.8 microgram/microliter, Roche), and 0.1 μl of Ribolock RNAse inhibitor (40 U/μl, Thermo Fisher) and 0.5 microliter of probe/primer mixes. This is referred to this subsequently as mastermix. This creates a total mix volume of 22 microliter, of which 20 microliters is used for droplet generation. Other concentrations of these components are likely to work as well.

Hydrolysis probe assays were designed to allow detection of amplified product. There were multiple iterations of primers and probes used, all being suitable for the assay. Some primers have been previously published, (BioTechniques 25:972-975 December 1998), but the assay is not dependent on these previously published primers and the disclosed set (ddPERT 3310) is superior in many characteristics (Table 2). Concentrations of primer in the final reaction were in the 225 to 450 nanomolar range and probe concentrations were between 125 and 250 nanomolar. Probes were labelled 5′ with either 6-FAM (6 fluoresceins) or HEX (Hexachlorofluorescein) fluorophores, had an internal Zen quencher and terminal Iowa Black quencher as supplied by Integrated DNA technologies (ZEN and Iowa Black are patent pending with IDT). Different qualities of these assays are discussed later. The use of MS2 as RNA template and MS2 specific primers can be substituted by other available RNA template with appropriate primers and probes.

TABLE 2

| Primer and probes | | | | | |
|---|---|---|---|---|---|
| purpose | name and | primer | Sequence (5′-3′) | modification | source |
| absolute | ddPERT | reverse | Ccactccgaagtgcgtataa | | self |
| or high | 3310 | primer 1 | (SEQ ID NO: 1) | | |
| | | forward | Tggttccatactggaggtga | | self |
| | | primer 1 | (SEQ ID NO: 2) | | |
| | | probe 1 | Cgacagcatgaattccgccg | fam zen | self |
| | | | (SEQ ID NO: 3) | iowablack | |
| Relative | ddPERT | reverse | Gccttagcagtgccctgtct | | published |
| or high | 1011 | primer 2 | (SEQ ID NO: 4) | | TM- |
| sensitivity | | | | | PERT |
| | | forward | Aacatgctcgagggcctta | | published |
| | | primer 2 | (SEQ ID NO: 5) | | TM- |
| | | probe 2 | Cagtgggatgctcctacatgtc | hex zen | self |
| | | | (SEQ ID NO: 6) | iowablack | |
| Relative | ddPERT | reverse | Gccttagcagtgccctgtct | | published |
| or high | ALT1011 | primer 2 | (SEQ ID NO: 7) | | TM- |
| | | forward | Aacatgctcgagggcctta | | published |
| | | primer 2 | (SEQ ID NO: 8) | | TM- |
| | | probe 3 | Aagcgttgacgctccctacg | fam zen | self |
| | | | (SEQ ID NO: 9) | iowablack | |
| cDNA | ddPERT | reverse | Ccactccgaagtgcgtataa | | self |
| length | LR | primer 1 | (SEQ ID NO: 10) | | |
| | | forward | Tggttccatactggaggtga | | self |
| | | primer 1 | (SEQ ID NO: 11) | | |
| | | probe 1 | Cgacagcatgaattccgccg | fam zen | self |
| | | | (SEQ ID NO: 12) | iowa black | |
| | | heat inactivation of RT followed by addition of | | | |
| | | reverse | Gccttagcagtgccctgtct | | published |
| | | primer 2 | (SEQ ID NO: 13) | | TM- |
| | | forward | Aacatgctcgagggcctta | | published |
| | | primer 2 | (SEQ ID NO: 14) | | TM- |
| | | probe 2 | Cagtgggatgctcctacatgtc | hex zen | self |
| | | | (SEQ ID NO: 15) | iowa black | |

### Carrying Out the Reactions Requires Several Phases.

### Pre-Anneal

Prior to the addition of the mastermix to the diluted RT standard or sample, it is beneficial to pre-anneal the MS2 RNA to the reverse primer from the assay. This is done by heating the mixture to 55° C. for 5 minutes and then cooling on ice. The length and temperature of this pre-annealing is not critical but is kept low enough to avoid denaturing the RNAse inhibitor.

### Mixing the Assay

Mastermix is combined 1:1 with lysed sample or recombinant standard to a total volume of 22 microliters. RT activity begins at the moment of mixing. Described herein is a single tube assay using the Bio-Rad mastermix, however there is no reason that the RT reaction could not take place in a buffer of one's choosing prior to addition of the Bio-Rad supermix (or other mix), provided that absolute quantification was not desired. This would be particularly useful if the effect of mix components on RT activity were the desired application.

### Reverse Transcription

The length of time and temperature at which the RT is able to create cDNA from MS2 phage can be highly variable and specific to the application and RT being tested. Assays were typically incubated from recombinant enzyme standards at up to 60 minutes at 42° C. This can occur prior to or after droplet generation. Incubations at 0-42° C. for 0-90 minutes is considered reasonable. Manual droplet generation on the Bio-Rad system takes roughly 1 minute to complete the generation of the droplets for each sample. This is the current minimum length of pre-droplet RT activity applicable to the absolute assay. This can be lengthened to anywhere up to 90 minutes in the high sensitivity assay and multiple time points can be compared. Post-droplet generation RT activity can take place at up to 42° C. for 60 minutes or more with no negative effect on the other reaction components. Intermediate lengths of the pre-droplet RT phase are recommended when comparing to an existing standard (relative RT activity) to maximize sensitivity as well as assay concentration range.

### RT Inactivation

In cases where an absolute assay is not being conducted, the use of an automated droplet generator is possible. However, the AutoDG from Bio-Rad completes only one row of eight samples at a time, requiring roughly 2 minutes to do so, which would result in a 24-30 minutes difference in incubation time of the RT reaction present in row 1 versus row 12. If this would negatively affect the interpretation of the results, RT inactivation is suggested. In one embodiment, incubation at 65° C. for 20 minutes prior to droplet generation was sufficient to stop the RT activity and did not negatively affect the other reaction components. Other means of terminating the reaction such as the addition of RNAse to degrade the MS2 are possible.

### Droplet Generation

Droplet generation can either occur in the manual or automatic droplet generator supplied by for example Bio-Rad. Each generates droplets 8 samples at a time using a microfluidic plate that pulls the mastermix through a proprietary Bio-Rad oil (FIG. 1), in the instant case Bio-Rad droplet generation oil for probes. Though there is also the availability of a droplet generation oil and reaction supermix for Evagreen dye, if that means of detection is desired.

In one embodiment of the absolute assay, the microfluidic chip would be modified to immediately combine separate diluted RT enzyme and the mastermix just prior to emulsification. This would require there to be two sample wells instead of one as is currently manufactured

The result of droplet generation is roughly 20,000 droplets from each 20 microliter sample input.

### PCR Cycling

42° C. for 0-90 minutes (post-droplet RT)

95° C. for 10 minutes (activates hotstart polymerase)
95° C. 30 seconds*
50-60 (generally 56) Celsius for 60 seconds* *repeated for 40-50 (generally 50) cycles

98° C. for 10 minutes

10° C. hold

All temperature changes occur at a rate of 2 degrees Celsius per second in a C1000 Bio-Rad thermocycler with 96 deepwell block.

### Reading the Droplets

Reading the droplets takes place on the Bio-Rad QX200 droplet reader. Each droplet is individually flowed past a detector where HEX, FAM or Evagreen fluorescence is read and the Poisson distribution is used to determine the number of DNA molecules or RT enzymes that were present, accounting for the fact that individual droplets are more likely to contain multiple molecules or enzymes as the input concentration increases. This is completed by QuantaSoft software provided by Bio-Rad on the QX200 and standalone PC. Concentrations are reported as copies per microliter of reaction or total positives per 20 microliter reaction. In this instance, copy refers to a single cDNA created by RT. All references to total positive droplets, total positives or total copies are referencing this same value, that is, the Poisson estimated total number of copies of cDNA in the 20 microliter reaction.

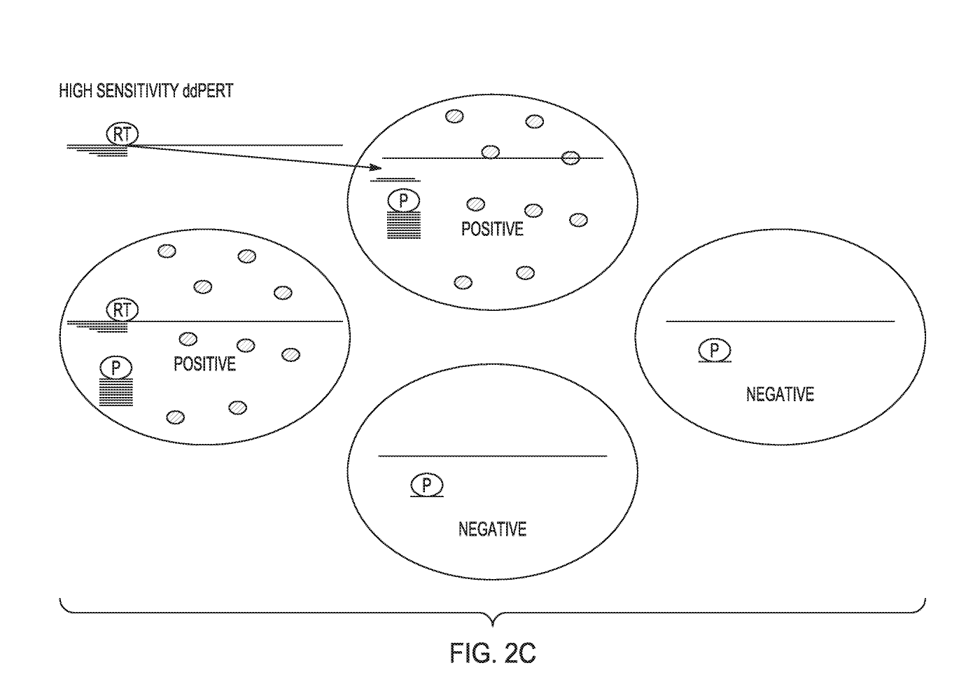

Typical results are shown in FIGS. 3 and 4. Each RT step was incubated for 60 minutes at 42° C. either prior to droplet generation in the case of ddPERT or prior to proceeding to the cycling for standard PERT. For M-MuLV, quantification distinguishable from background is valid down to 10 picoUnits, whereas traditional PERT is only usable to 1 nanoUnit, correlating to cycle 35 of qPCR. This is a 100-fold difference in sensitivity. For AMV RT, quantification distinguishable from background is valid down to 1 nanoUnit, whereas traditional PERT is only usable to 10 nanoUnits, correlating to cycle 35 of qPCR. This is a 10-fold difference in sensitivity. Previous publications by Arifa Khan STF-PERT (J Virol Methods. 2003 Mar; 108(1):139-42.) claim, but do not show that 1 nanoU is detectable by that variant of PERT.

FIG. 3 shows the comparison of ddPERT to standard PERT for duplicate standards. Results for M-MuLV are shown in A and AMV is shown in B. On the left for each panel is the average number of positives in the total reaction calculated by Bio-Rad ddPCR software. On the right for each panel is the raw data for duplicate samples taken from the cfx384 thermocycler. Labelled arrows indicate what we are defining as the useful range for traditional PERT. Usable ranges of the assay for each standard are indicated by labelled arrows.

Of note was that there were differences in the amount of amplification that occurred in the RT reaction for these enzymes. FIG. 4 shows how much this can contribute to the total signal generated and explains why current PERT assays are not absolutely quantitative. As shown, the difference between absolute quantification and 60 minutes time course is roughly 25-fold.

FIG. 4 provides a time course of 625 nanoUnits of M-MuLV recombinant standard. Duplicate reactions containing the same input where incubated at 42° C. at times stated prior to droplet generation. Additional RT incubation did not take place inside the droplet prior to heating to 95° C. to activate the polymerase, which explains the low amount present at 0 minutes. By contrast the absolute sample was incubated at 42° C. for 60 minutes after droplet generation. The extreme variability of the pre-droplet incubated sample signal over time demonstrates the amount of amplification that can occur at this phase, thereby preventing typical PERT from being absolutely quantitative.

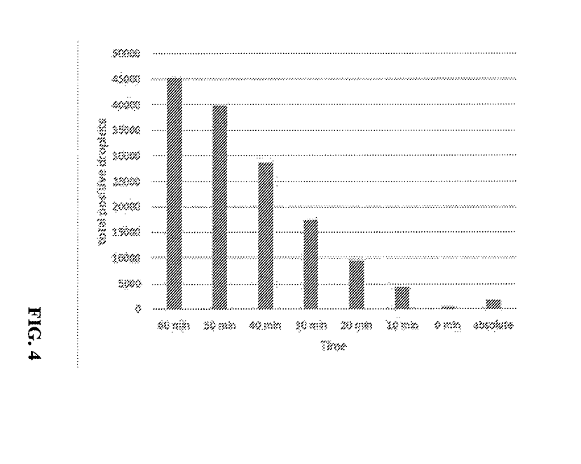

FIG. 5 provides a schematic for the redesigned PERT assay. Primers and probes are listed as ddPERT LR in Table 2. Reverse transcriptase (RT), Polymerase (P) MS2 RNA (blue line), primers (blue arrows). Hydrolysis probes with FAM labeling (blue) or HEX (green), Internal zen quencher (grey) Iowa black dark quencher (black). Fluorescence is released from the quencher by the actions of polymerase that hydrolyses the probe.

The primer and hydrolysis probe assay were designed to be located more towards the 3′ side of MS2 phage (FIG. 5). This is referred to it as 3310 as that is the genomic location of the rightmost base in this assay. This location takes advantage of the fact that RT travels from right to left on the RNA template. For some RTs this may delay the generation of additional cDNAs, as the RT may not be prone to template switching prior to reaching the 5′ end of the template. This assay decreased the additional signal in the M-MuLV absolute reaction by nearly 50% (not shown). This knowledge will allow one to even further develop an optimized primer and probe set for each application, as there is additional designable area in the 5′ and 3′ regions of MS2 for the design of sensitive versus absolute assays respectively.

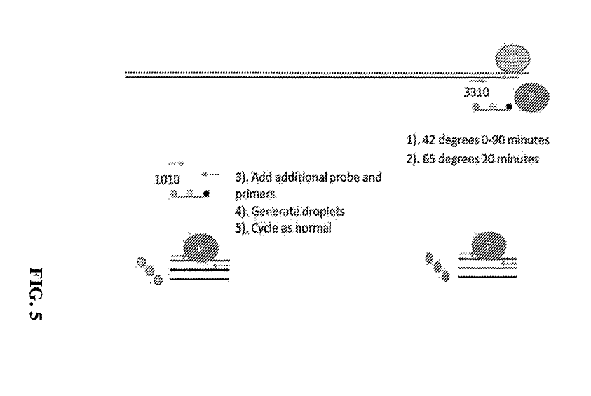

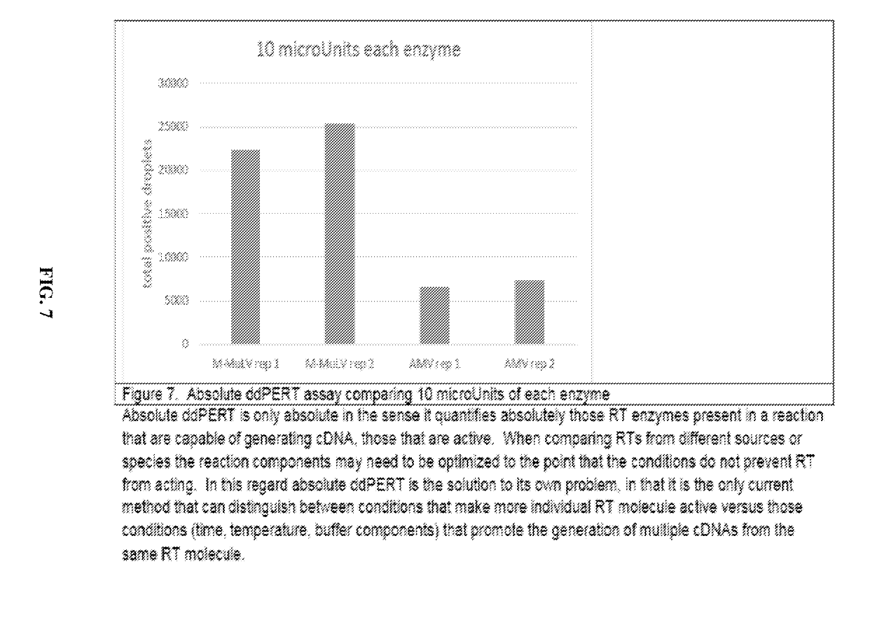

This assay was to be used with existing assays and the conditions above to measure the amount of long cDNAs being created. Existing assays are around nucleotide 1011 at their leftmost location. They are added after RT inactivation to prevent cDNA from being created from the reverse primer of this assay. 200 nanoUnits of M-MuLV and Avian Myeloblastosis Virus (AMV) (NEB part number M0277) reverse transcriptases were comparted, results depicted in FIG. 6. Absolute assays for each enzyme indicated a mere 2-3.5 fold more M-MuLV than AMV at concentrations bracketing the concentration tested. A sample of those results are indicated in FIG. 7.

It can be clearly seen that M-MuLV RT completes additional cDNAs at a much faster rate over time than AMV, whereas AMV is more likely to complete a cDNA that covers the entire range of both assays. This further shows the drawback to typical PERT in that different RTs show different rates of signal amplification and are therefore not comparable.

Both are defined commercially with one unit as the amount of enzyme required to incorporate 1 nmol of dTTP into an acid-insoluble form in 10 minutes at 37° C. using poly(rA)-oligo(dT) as template primer, though with potentially different reaction conditions. Knowing both this measure and an absolute count by ddPERT allows one to determine how much activity is present in single RT molecules and to compare the activity of those with different structures.

This also demonstrates an additional use of ddPERT. The separating of the RT and polymerase reactions, which allows one to more clearly define everything present in the RT reaction, allows for the testing of additives and buffer compositions that promote the generation of long cDNAs. It also allows for the testing of recombinant RTs with amino acid substitutions that would enable the creation or identification of RTs with the ability to create long cDNA in silico, which is highly desired in modern biological applications. RNA templates with different properties, such as base composition considered difficult for RT processivity could also be selected in order to screen RT properties in this assay.

### Bibliography

BioTechniques 25(6):972-975 (1998).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

1. A method to determine if a sample comprises a reverse transcriptase comprising:
a) mix a sample with template RNA, along with nucleotides, primer, and DNA polymerase to generate a mixture;
b) form droplets from the mixture generated in a);
c) allow reverse transcription to occur in the mixture of a) prior to or after droplet formation in b);
d) amplify any cDNA in said droplet of b) or c); and
e) detect for the presence of cDNA;
wherein reverse transcriptase is present in the sample if cDNA is detected in e) and the absence of cDNA in e) relates to an absence of reverse transcriptase.

2. The method of claim 1, wherein sample has been exposed to conditions effective to lyse viral particles.

3. The method of claim 1, wherein one or more probes are added to the mixture of a).

4. The method of claim 3, where in the probe is labeled.

5. The method of claim 4, where the label is fluorescent.

6. The method of claim 1, wherein the primer comprises a forward and a reverse primer. (Original) The method of claim 6, wherein the reverse primer is incubated with the template RNA prior to addition to the mixture of a).

8. The method of claim 1, wherein an RNAse inhibitor is added to the mixture of a).

9. The method of claim 1, wherein the amplification in d) comprises a polymerase chain reaction.

10. The method of claim 1, further comprising quantifying reverse transcriptase enzyme or viral production present in said sample from the cDNA present in e).

11. A method to screen for a reverse transcriptase inhibitor comprising:
a) prepare a mixture of template RNA, nucleotides, reverse transcriptase, primer, DNA polymerase, and a possible reverse transcriptase inhibitor agent;
b) form droplets from the mixture generated in a);
c) allow reverse transcription to occur in the mixture of a) prior to or after droplet formation in b);
d) amplify any cDNA in said droplet of b) or c); and
e) detect the presence of cDNA in d);
if no cDNA is detected in e), then said agent is a reverse transcriptase inhibitor.

12. A method to screen for a reverse transcriptase with resistance to an inhibitor comprising:
a) prepare a mixture of template RNA, nucleotides, reverse transcriptase, primer, DNA polymerase, and a reverse transcriptase inhibitor;
b) form droplets from the mixture generated in a);
c) allow reverse transcription to occur in the mixture of a) prior to or after droplet formation in b);
d) amplify any cDNA in said droplet of b) or c); and
e) detect the presence of cDNA in d);
if cDNA is detected in e), then said reverse transcriptase is resistant to said inhibitor.

13. The method of claim 11, wherein one or more probes are added to the mixture of a).

14. The method of claim 13, where in the probe is labeled.

15. The method of claim 14, where the label fluorescent.

16. The method of claim 11, wherein the primer comprises a forward and a reverse primer.

17. The method of claim 16, wherein the reverse primer is incubated with the template RNA prior to addition to the mixture of a).

18. The method of claim 11, wherein an RNAse inhibitor is added to the mixture of a).

19. The method of claim 11, wherein the amplification in d) comprises a polymerase chain reaction.


SEQUENCE LISTING (19 sequences)

SEQ ID NO: 1 - DNA, 20 bp, Artificial Sequence
  A synthetic oligonucleotide primer
ccactccgaagtgcgtataa

SEQ ID NO: 2 - DNA, 20 bp, Artificial Sequence
  A synthetic oligonucleotide primer
tggttccatactggaggtga

SEQ ID NO: 3 - DNA, 20 bp, Artificial Sequence
  A synthetic oligonucleotide primer
cgacagcatgaattccgccg

SEQ ID NO: 4 - DNA, 20 bp, Artificial Sequence
  A synthetic oligonucleotide primer
gccttagcagtgccctgtct

SEQ ID NO: 5 - DNA, 19 bp, Artificial Sequence
  A synthetic oligonucleotide primer
aacatgctcgagggcctta

SEQ ID NO: 6 - DNA, 22 bp, Artificial Sequence
  A synthetic oligonucleotide primer
cagtgggatgctcctacatgtc

SEQ ID NO: 7 - DNA, 20 bp, Artificial Sequence
  A synthetic oligonucleotide primer
gccttagcagtgccctgtct

SEQ ID NO: 8 - DNA, 19 bp, Artificial Sequence
  A synthetic oligonucleotide primer
aacatgctcgagggcctta

SEQ ID NO: 9 - DNA, 20 bp, Artificial Sequence
  A synthetic oligonucleotide primer
aagcgttgacgctccctacg

SEQ ID NO: 10 - DNA, 20 bp, Artificial Sequence
  A synthetic oligonucleotide primer
ccactccgaagtgcgtataa

SEQ ID NO: 11 - DNA, 20 bp, Artificial Sequence
  A synthetic oligonucleotide primer
tggttccatactggaggtga

SEQ ID NO: 12 - DNA, 20 bp, Artificial Sequence
  A synthetic oligonucleotide primer
cgacagcatgaattccgccg

SEQ ID NO: 13 - DNA, 20 bp, Artificial Sequence
  A synthetic oligonucleotide primer
gccttagcagtgccctgtct

SEQ ID NO: 14 - DNA, 19 bp, Artificial Sequence
  A synthetic oligonucleotide primer
aacatgctcgagggcctta

SEQ ID NO: 15 - DNA, 22 bp, Artificial Sequence
  A synthetic oligonucleotide primer
cagtgggatgctcctacatgtc

SEQ ID NO: 16 - DNA, 20 bp, Artificial Sequence
  A synthetic oligonucleotide primer
gccttagcagtgccctgtct

SEQ ID NO: 17 - DNA, 19 bp, Artificial Sequence
  A synthetic oligonucleotide primer
aacatgctcgagggcctta

SEQ ID NO: 18 - DNA, 22 bp, Artificial Sequence
  A synthetic oligonucleotide primer
cagtgggatgctcctacatgtc

SEQ ID NO: 19 - DNA, 20 bp, Artificial Sequence
  A synthetic oligonucleotide primer
aagcgttgacgctccctacg